(19) United States
(12) Patent Application Publication
(10) Pub. No.: US 2007/0212408 A1
(43) Pub. Date: Sep. 13, 2007

(54) Stable Pharmaceutical Composition Containing Benzimidazole Derivatives and Method of Manufacturing the Same

(76) Inventors: Nam Ho Kim, Gyeonggi-do (KR); Jin Young Choi, Gyeonggi-do (KR); Jae Sun Kim, Gyeonggi-do (KR); Nam Kyu Lee, Gyeonggi-do (KR); Je Ho Ryu, Seoul (KR); Yong Youn Hwang, Gyeonggi-do (KR); Yong Ho Oh, Gyeonggi-do (KR); Dong Sun Min, Gyeonggi-do (KR); Key An Um, Gyeonggi-do (KR); Wei-Jong Kwak, Seoul (KR); Do Seung Kum, Seoul (KR)

Correspondence Address: Ronald R Santucci;Frommer Lawrence Haug, 745 Fifth Avenue, New York, NY, 10151, US

(21) Appl. No.: 11/587,720

(22) PCT Filed: Apr. 27, 2005
(86) PCT No.: PCT/KR05/01214

§ 371 Date: Apr. 16, 2007

(30) Foreign Application Priority Data
Apr. 30, 2004 (KR) 10-2004-0030583

(51) Int. Cl. A61K 31/724; C08B 30/18; A61K 9/48; A61K 9/20

(52) U.S. Cl. 424/451; 514/58; 536/46; 424/464

(57) ABSTRACT

The present invention relates to an inclusion complex containing a benzimidazole derivative with excellent storage stability and a method of its preparation. In particular, the present invention relates to an inclusion complex containing a benzimidazole derivative with improved storage stability and a method of its preparation, where an inclusion complex is manufactured by performing an inclusion reaction by combining a benzimidazole derivative, cyclodextrin and a water-soluble polymer in an aqueous alkali solution in order to be formulated after stabilizing an acid-unstable benzimidazole derivative.

### TECHNICAL FIELD

The present invention relates to an inclusion complex containing a benzimidazole derivative with excellent storage stability and a method of its preparation. In particular, the present invention relates to an inclusion complex containing a benzimidazole derivative with improved storage stability and a method of its preparation, where an inclusion complex is manufactured by performing an inclusion reaction by combining a benzimidazole derivative, cyclodextrin and a water soluble polymer in an aqueous alkali solution in order to be formulated after stabilizing an acid-unstable benzimidazole derivative.

### BACKGROUND OF INVENTION

In general, benzimidazole derivatives that inhibit secretion of gastric acid are highly unstable under acidic and basic conditions, and thus easily undergo color change and decomposition. For example, the half-life of omeprazole is less than 10 min in an acidic condition, 14 hrs at pH 7, and about 300 days in an alkali condition of pH 11 (Pilbrant A and Cederberg C, Scand J. Gastroenterology, Suppl. 108, 113-120(1985)). Therefore, in preparations of benzimidazole derivatives for oral administration, it is essential that the preparations are not in contact with gastric acid so that they can be delivered to small intestine without being decomposed in stomach and also they should contain alkali components in the core where the drug is contained in order to increase storage stability.

For stable preparation of an acid-unstable compound, it is essential that the stability of the compound itself along with the stability of the preparation be secured. Further, the stability during the preparation process and in the body after administration as well as rapid absorption in small intestine should be considered.

Korean Pat. No. 87-9718 discloses a technique where a core is formed by mixing omeprazole with an alkali material and a water-soluble inner coated layer is formed thereto followed by enteric coating.

Korean Pat. No. 91-4579 discloses a technique where a core containing an omeprazole mixed with an alkali reaction compound or an alkali salt of omeprazole readily miscible with an alkali reaction compound is coated with at least one inert inner layer and then further coated with enteric coating, thereby manufacturing a preparation for oral administration.

However, the above-mentioned methods are not advantageous in that their coating process is very complex. Further, the water soluble inner coated layer of the above preparations, after they are administered, become partially dissolved in the stomach by the gastric acid, which is diffused through the enteric coating from the inner side of the stomach, and the gastric acid also penetrates the core and dissolves the alkali material and the resulting dissolved alkali material in turn partially destroys the enteric coating. As a result, omeprazole is discolored and decomposed while the preparations are staying in the stomach, and thus the stability of the preparations are not fully secured.

Korean Pat. No. 96-8231 discloses a technique where an acid unstable compound is stabilized by using cyclodextrin and the oral preparation is manufactured using an inclusion complex without containing an alkali material.

WO Publication No. 98-40069 discloses a technique where benzimidazole compounds are stabilized by using cyclodextrin and amino acids. However, the method is only useful to omeprazole among benzimidazole derivatives because it has a lot of limitations in its application to other derivatives such as lansoprazole.

The inventors of the present invention attempted to manufacture the inclusion complex of lansoprazole according to the methods disclosed in the above patents but failed to precipitate it out even with cooling after evaporatation under reduced pressure in an alkali solution. Further, considering that precipitaction may not be possible due to the difference in solubility according to pH even though inclusion is formed, it was attempted to neutralize the inclusion mixture with a weak acid and cooled down to induce the solidification. However, thus obtained solid was not an inclusion complex and also did not show a good result in stability test. This is because each benzimidazole derivative has a different substituent and then a different structure may lead to the difference in inclusion rate.

To resolve the above problem due to difference in structure it was necessary to develop a method to optimize the efficiency of inclusion in an aqueous solution. Accordingly, the inventors of the present invention developed a novel method by adding a water-soluble polymer to a cyclodextrin solution as a way to expedite the inclusion reaction for the benzimidazole derivative, a drug which is known hard to form an inclusion complex from its structural point of view and completed this invention. Therefore, an object of this invention is to provide an inclusion complex containing a benzimidazole derivative with greatly improved storage stability and a method of its preparation.

### DETAILED DESCRIPTION OF INVENTION

The present invention relates to an inclusion complex comprising benzimidazole with improved storage stability wherein a water-soluble polymer is added during inclusion reaction of a benzimidazole derivative into cyclodextrin, and its pharmaceutical preparations.

The present invention also relates to a method of manufacturing an inclusion complex comprising a benzimidazole derivative comprising:

a) combining a benzimidazole derivative, cyclodextrin and a water-soluble polymer in an aqueous alkali solution;

b) stirring the above mixture at about 20 to 100° C. and adjusting its pH to about 7.0 to 11.0; and

c) cooling the mixture down to about 0 to 30° C., filtering, washing and drying of the mixture to manufacture an inclusion complex.

The present invention is described further in detail hereunder.

In an embodiment of the present invention, benzimidazole derivatives are stabilized by using cyclodextrin. The benzimidazole derivatives are reacted in an aqueous alkali solution containing a water-soluble polymer, but the inclusion complex obtained as a result does not contain any alkali component.

Herein below is disclosed a method for manufacturing an inclusion complex comprising a benzimidazole derivative.

In the first step, a mixture is prepared by combining a benzimidazole derivative, cyclodextrin and a water-soluble polymer in an aqueous alkali solution. In examples of the present invention, only lansoprazole and omeprazole are disclosed but pantoprazole, timoprazole, picoprazole, rabeprazole and the like can be also used in the present invention.

Cyclodextrins in general have a certain size of hydrophobic cavities in their structures and thus hydrophobic compounds can be included into the cavities to protect the compounds from the outside environment. Cyclodextrins are grouped into α-cyclodextrin, β-cyclodextrin, and γ-cyclodextrin according to size and properties. In the present invention, cyclodextrins to be used are all kinds of cyclodextrins including the above-mentioned 3 kinds of cyclodextrins, preferably β-cyclodextrins or their derivatives with cavities in the range of from about 6.0 to 6.5 Å. The cyclodextrin is preferably used in the amount of from about 1.0 to 5.0 moles with reference to 1 mol of the benzimidazole derivative, more preferably from about 2.0 to 3.0 moles. If the cyclodextrin is used less than 1 mole there will be acid-unstable compound remaining unincluded. On the other hand, if it is used more than 5 moles the content of inclusion complex will be decreased due to the presence of excessive cyclodextrins, which remain unreacted.

In another embodiment of the present invention, a water-soluble polymer is used to increase solubility and stability in a given reaction solution and to expedite inclusion reaction by interacting with cyclodextrin. The water soluble polymer to be used in the present invention is at least one selected from the group consisting of polyethyleneglycol(PEG), polyvinylpyrrolidinon(PVP), carboxymethylcellulose(CMC), hydroxypropylcellulose(HPC), hydroxymethylcellulose(HMC), hydroxyethylcellulose(HEC), hydroxypropylmethylcellulose(HPMC) and hydroxypropylethylcellulose(HPEC). The water-soluble polymer is preferably used in the amount of from about 0.1 to 100 parts by weight with reference to 100 parts by weight of a benzimidazole derivative, more preferably from about 1.0 to 50 parts by weight. If the water-soluble polymer is used less than 0.1 parts by weight, the desired stabilization will not be obtained. In contrast, if it exceeds 100 parts by weight, this will result in drastic increase in viscosity of the reaction mixture, which leads to incomplete formation of inculsion complex, difficulties in washing and filtration, thereby resulting in an extremely low yield of inclusion complex.

As for the aqueous alkali solution to be used in the present invention, it can be one or a mixture of at least two selected from the group consisting of alkali metal hydroxides, inorganic or organic alkali salts, amines and buffer solutions.

Here, examples of the alkali metal hydroxides are sodium hydroxide, potassium hydroxide, barium hydroxide, and calcium hydroxide. Examples of the inorganic alkali salts are sodium salts of boric acid, carbonic acid or phosphoric acid. Examples of the organic alkali salts are sodium acetate or sodium citrate.

Amines are selected from the group consisting of diethylamine, triethylamine, butylamine, ethylenediamine, triethanolamine, propylamine, dipropylamine, diethanolamine, monoethanolamine, isobutylamine, diisopropylamine, tert-butylamine, dibutylamine, diisobutylamine, tributylamine, pentylamine, and dipentylamine.

As for the buffer solution, it is preferable to use one of buffer solutions containing carbonate, phosphate, amine salt or borate.

In the second step, an inclusion complex is solidified from the above mixture after stirring under the heat and adjusting the pH of the mixture to about pH 7.0 to 11.0.

The above stirring is performed at about from 20 to 100° C., preferably 40 to 80° C. For the above pH adjustment to about pH 7.0 to 11.0, it is preferable to use at least one organic or inorganic material having pKa in the range of about 2.0 to 10.0, more preferably boric acid, acetic acid or ammonium chloride. If the temperature for stirring is below 20° C. it will increase the amount of a solvent to be used for dissolving cyclodextrins and drugs. In contrast, if it exceeds 100° C. it will result in decomposition of the drug.

In the third step, the above reaction mixture is manufactured into an inclusion complex by passing it through processes of cooling, filtration, washing, and drying. The cooling process is performed at about 0 to 30° C., preferably about 0 to 10° C. If the cooling temperature is lower than 0° C., it results in over-cooling of the reaction mixture thus leading to concommitant precipitations of unincluded cyclodextrin or impurities along with inclusion complex. If the temperature exceeds 30° C., however, it results in a marked decrease in yield. The final inclusion complex is obtained by washing the resulting filtrate several times with a small amount of cold water to remove alkali components followed by drying.

Thus obtained inclusion complexes can be stored for a relatively long period of time by securing superior storage stability of the starting materials on temperature and humidity, and they can be ultimately formulated into tablets, capsules, and the like while not being decomposed by the influences of the temperature and humidity conditions during the manufacturing process.

In addition, the final inclusion complex does not contain any alkali components because these alkali components only serve as a reaction mediator and their purposes or actions are quite different from the conventional alkalinizing agent, which is present as a core component.

This invention is explained in more detail based on the following Examples, however, they should not be construed as limiting the scope of this invention.

### EXAMPLES

### Example 1-5

A mixture of lansoprazole (369 mg, 1 mmol) and β-cyclodextrin (2.56 g, 2.2 mmol) was added with hydroxypropylmethylcellulose in the amount of 20, 50, 100, 150 and 200 mg, respectively, as shown in the following table 1 and then added with 30 mL of distilled water. The reaction mixture was added with 1.2 mL of 1M-NaOH and then stirred at 50° C. for 6 hrs. Then, 74 mg of boric acid dissolved in 2.22 mL of distilled water was added thereto and stirred at 50° C. for 10 min. The above reaction mixture was cooled down to 5° C. and kept in that condition for 18 hrs to form an inclusion complex. The inclusion complex was then filtered, washed several times with cold distilled water and then dried under vacuum at 40° C. for 12 hrs to finally obtain a white inclusion complex, respectively.

TABLE 1

Content of hydroxypropylmethylcellulose

| Classification | mg | wt. parts* |
|---|---|---|
| Example 1 | 20 | 5.42 |
| Example 2 | 50 | 13.6 |
| Example 3 | 100 | 27.1 |
| Example 4 | 150 | 40.7 |
| Example 5 | 200 | 54.2 |

*based on 100 wt. parts of lansoprazole

### Comparative Example 1

Experiment was performed the same as in example 1 except that hydroxypropylmethylcellulose was not used.

### Comparative Example 2

A mixture of lansoprazole (369 mg, 1 mmol) and β-cyclodextrin (2.56 g, 2.2 mmol) was added with 100 mg of hydroxypropylmethylcellulose, which is 27.1 parts by weight with reference to 100 parts by weight of lansoprazole. The mixture was then uniformly ground by using a mortar, sifted and then dried at 40° C. under vacuum for 12 hrs to obtain a white mixture.

### Test Example 1

### Storage Stability Test of Inclusion Complexes Containing Lansoprazole

Storage stability tests were performed at 60° C., 75% RH on inclusion complexes obtained in examples 1-5, comparative examples 1 and 2, and lansoprazole itself and the relative content compared to that of the initial time according to time passage was measured using HPLC.

TABLE 2

| | Content | | | |
|---|---|---|---|---|
| | after | Content after | Content after | Color Change |
| Classification | 1 wk (%) | 2 wk (%) | 4 wk (%) | after 4 wk |
| lansoprazole | 96.62 | 76.88 | Not detected | Dark brown |
| Example 1 | 97.0 | 92.89 | 94.54 | White |
| Example 2 | 105.61 | 101.38 | 98.66 | White |
| Example 3 | 100.42 | 95.91 | 92.86 | White |
| Example 4 | 100.05 | 97.44 | 95.87 | White |
| Example 5 | 106.02 | 100.33 | 90.35 | White |
| Comp. Ex. 1 | 91.53 | 73.95 | Not detected | Brown |
| Comp. Ex. 2 | 91.86 | 80.42 | 27.68 | Brown |

N.B. Content (%) is indicated with reference to that of initial time.

As shown in the above table 2, the inclusion complexes obtained in examples 1-5 of the present invention are shown to have superior storage stability as compared with the inclusion complexes and lansoprazole obtained in comparative examples.

### Example 6

A mixture of omeprazole (345 mg, 1 mmol) and β-cyclodextrin (2.56 g, 2.2 mmol) was added with 50 mg of hydroxypropylmethylcellulose, which is 14.5 parts by weight with reference to 100 parts by weight of omeprazole, and then further added with 30 mL of distilled water. The reaction mixture was added with 1.2 mL of 1M-NaOH and then stirred at 50° C. for 1 hr. Then, 74 mg of boric acid dissolved in 2.22 mL of distilled water was added thereto and stirred at 50° C. for 10 min. The above reaction mixture was cooled down to 5° C. and kept in that condition for 18 hrs to form an inclusion complex. The inclusion complex was then filtered, washed several times with cold distilled water and then dried under vacuum at 40° C. for 12 hrs to finally obtain a white inclusion complex.

### Test Example 2

### Storage Stability Test of Inclusion Complexes Containing Omeprazole

Storage stability tests were performed at 60° C., 75% RH on the inclusion complex obtained in example 6 and omeprazole itself and the content compared to that of the initial time according to time passage was measured using HPLC.

TABLE 3

| | Content after | Content after |
|---|---|---|
| Classification | 1 wk (%) | 2 wk (%) |
| omeprazole | 75.1 | 0 |
| Example 6 | 97.5 | 95.9 |

As shown in the above table 3, the inclusion complex obtained in example 6 of the present invention is shown to have greatly improved storage stability.

### Example 7

A mixture of lansoprazole (369 mg, 1 mmol) and β-cyclodextrin (2.56 g, 2.2 mmol) was added with 200 mg of polyvinylpyrrolidinone, which is 54.2 parts by weight with reference to 100 parts by weight of lansoprazole, and then further added with 30 mL of distilled water. The reaction mixture was added with 1.2 mL of 1M-NaOH and then stirred at 50° C. for 6 hr. Then, 74 mg of boric acid dissolved in 2.22 mL of distilled water was added thereto and stirred at 50° C. for 10 min. The above reaction mixture was cooled down to 5° C. and kept in that condition for 18 hrs to form an inclusion complex. The inclusion complex was then filtered, washed several times with cold distilled water and then dried under vacuum at 40° C. for 12 hrs to finally obtain a white inclusion complex.

### Example 8

A mixture of lansoprazole (369 mg, 1 mmol) and β-cyclodextrin (2.56 g, 2.2 mmol) was added with 50 mg of carboxylmethylcellulose, which is 13.6 parts by weight with reference to 100 parts by weight of lansoprazole, and then further added with 30 mL of distilled water. The reaction mixture was added with 1.2 mL of 1M-NaOH and then stirred at 50° C. for 6 hr. Then, 74 mg of boric acid dissolved in 2.22 mL of distilled water was added thereto and stirred at 50° C. for 10 min. The above reaction mixture was cooled down to 5° C. and kept in that condition for 18 hrs to form an inclusion complex. The inclusion complex was then filtered, washed several times with cold distilled water and then dried under vacuum at 40° C. for 12 hrs to finally obtain a white inclusion complex.

### Test Example 3

### Storage Stability Test of Inclusion Complexes Containing Lansoprazole

Storage stability tests were performed at 60° C., 75% RH on inclusion complexes obtained in examples 7 and 8 and lansoprazole itself, and the content compared to that of the initial time according to time passage was measured using HPLC.

TABLE 4

| | Content after | Content after |
|---|---|---|
| Classification | 1 wk (%) | 2 wk (%) |
| lansoprazole | 96.62 | 76.88 |
| Example 7 | 102.98 | 103.01 |
| Example 8 | 96.68 | 91.50 |

As shown in the above table 4, the inclusion complexes obtained in examples 7 and 8 of the present invention are shown to have greatly improved storage stability.

### INDUSTRIAL APPLICABILITY

As stated above, the inclusion complexes containing benzimidazole derivatives of the present invention can be stored for a relatively long period of time by securing superior storage stability of the starting materials on temperature and humidity conditions. In addition, they can be easily formulated into tablets, capsules, and the like while not being decomposed by the influences of the temperature and humidity conditions during the manufacturing process. Moreover, the final inclusion complex does not contain any alkali components because these alkali components only serve as a reaction mediator and their purposes or actions are quite different from the conventional alkalinizing agent, which is present as a core component.

The invention has been described in detail with reference to preferred embodiments thereof. However, it will be appreciated that those skilled in the art, upon consideration of the disclosure, may make modifications and improvements within the scope and spirit of the invention.

1. An inclusion complex comprising benzimidazole with improved storage stability wherein a water-soluble polymer is added when a benzimidazole derivative is being included into cyclodextrin and is used in the amount of about 0.1 to 100 parts by weight with reference to 100 parts by weight of said benzimidazole derivative.

2. In claim 1, said benzimidazole derivative is selected from the group consisting of lansoprazole, omeprazole, pantoprazole, timoprazole, picoprazole and rabeprazole.

3. In claim 1, cyclodextrin is used in the amount of about 1 to 5 moles with reference to 1 mole of said benzimidazole derivative.

4. (canceled)

5. In claim 1, said cyclodextrin is β-cyclodextrin or its derivative.

6. In claim 1, said water-soluble polymer is at least one selected from the group consisting of polyethyleneglycol, polyvinylpyrrolidinon, carboxymethylcellulose, hydroxypropylcellulose, hydroxymethylcellulose, hydroxyethylcellulose, hydroxypropylmethylcellulose and hydroxypropylethylcellulose.

7. A pharmaceutical preparation comprising the inclusion complex in claim 1 and pharmaceutically acceptable ingredients.

8. The pharmaceutical preparation according to claim 7, wherein said inclusion complex is formulated in the form of a tablet or a capsule.

9. A method of manufacturing an inclusion complex comprising a benzimidazole derivative comprising:
a) combining a benzimidazole derivative, cyclodextrin and a water soluble polymer in an aqueous alkali solution; and
b) stirring said mixture at about 20 to 100° C. and adjusting its pH to about 7.0 to 11.0; and
c) cooling down said mixture to about 0 to 30° C., filtering, washing and drying of said mixture to manufacture an inclusion complex.

10. In claim 9, said aqueous alkali solution is selected form the group consisting of alkali metal hydroxides, inorganic or organic alkali salts, amines and a buffer solution.

11. In claim 9, said pH is adjusted by using at least one selected from an organic or inorganic material in the range of about pKa 2.0 to 10.0.
